(19) United States
(12) Patent Application Publication
(10) Pub. No.: US 2020/0399587 A1
(43) Pub. Date: Dec. 24, 2020

(54) CULTURE MEDIUM FOR IMPROVING DEVELOPMENT OF BOVINE IN VITRO FERTILIZED EMBRYOS AND CLONED EMBRYOS

(71) Applicant: NORTHEAST A&F UNIVERSITY, xianyang (CN)

(72) Inventors: Jingcheng ZHANG, xianyang (CN); YUEMENG HUANG, xianyang (CN); MIN ZHANG, xianyang (CN); DEBAO WANG, xianyang (CN); YONG WANG, xianyang (CN); YONG ZHANG, xianyang (CN)

(73) Assignee: NORTHEAST A&F UNIVERSITY

(21) Appl. No.: 17/008,676

(22) Filed: Sep. 1, 2020

Related U.S. Application Data
(63) Continuation of application No. PCT/CN2020/100821, filed on Jul. 8, 2020.

(30) Foreign Application Priority Data
May 9, 2020 (CN) 202010387376.8

(51) Int. Cl. C12N 5/00

(52) U.S. Cl. CPC C12N 5/0018; C12N 2501/105; C12N 2501/11; C12N 2500/38; C12N 2523/00; C12N 2501/235; C12N 2501/119

(57) ABSTRACT

A culture medium for improving the development of bovine in vitro fertilized embryos and cloned embryos includes a SOF basal medium, progesterone, vitamin E, L-carnitine, FGF, corticosterone, citrulline, EGF, IGF and LIF.

### TECHNICAL FIELD

This disclosure relates to embryo engineering for livestock, and more particularly to a culture medium for improving the development of bovine in vitro fertilized embryos and cloned embryos.

### BACKGROUND

In vitro culture of livestock embryos has great potential in research and commercial applications, and can be widely applied in breeding of cattle individuals or herds with high genetic value, and breeding of animals by gene editing. However, the embryos obtained from in vitro culture are often inferior to, in quality and quantity, embryos that develop in vivo. For example, the embryos obtained from in vitro culture often experience delayed embryonic development in the compaction stage, and also suffer oxidative stress and insufficient growth, which results in a gradual decline in embryo cleavage rate and a decrease in the number of cells developing into blastocysts, affecting the embryo quality and the in vitro development rate of early embryos. Therefore, the applications of such embryo in vitro culture technique are greatly limited.

Given the above, there is an urgent need for those skilled in the art to develop a culture medium for improving the development of bovine in vitro fertilized embryos and cloned embryos.

### SUMMARY

In view of this, an object of this application is to provide a culture medium for improving the development of bovine in vitro fertilized embryos and cloned embryos, which can effectively boost the development rate and quality of bovine in vitro fertilized embryos and somatic cell cloned embryos.

Technical solutions of this application are described as follows.

In a first aspect, this application provides a culture medium for improving the development of bovine in vitro fertilized embryos and cloned embryos, comprising: a synthetic oviduct fluid (SOF) basal medium, citrulline, progesterone, vitamin E, L-carnitine, fibroblast growth factor (FGF), corticosterone, epidermal growth factor (EGF), insulin growth factor (IGF) and leukemia inhibitory factor (LIF).

Progesterone and corticosterone can better maintain the in vitro survival and proliferation of embryonic cells; vitamin E can alleviate the adverse effects of reactive oxygen species (ROS) on cells; L-carnitine can promote the fat metabolism in the embryos to produce energy; citrulline plays a role in regulating intracellular NO signal to keep the nitrogen level stable; and FGF, EGF, IGF and LIF all show an effect in promoting the cell growth and proliferation and improving cell morphology and cell viability.

The introduction of these nine additives to a basal medium can effectively reduce the oxidative damage of bovine in vitro fertilized embryos and improve the development rate and quality of bovine in vitro fertilized embryos.

In an embodiment, the culture medium comprises 1-100 ng/mL of the progesterone, 10-1000 μM of vitamin E, 0.1-30 mM of L-carnitine, 1-100 ng/mL of FGF, 1-100 ng/mL of corticosterone, 1-100 ng/mL of EGF, 1-100 ng/mL of IGF and 1-100 ng/mL of LIF.

In an embodiment, the culture medium comprises 1-10 ng/mL of the progesterone, 10-100 μM of vitamin E, 1-3 mM of L-carnitine, 10-100 ng/mL of FGF, 1-10 ng/mL of corticosterone, 0.01-1 mg/mL of citrulline, 1-10 ng/mL of EGF, 10-100 ng/mL of IGF and 10-100 ng/mL of LIF.

In an embodiment, the culture medium further comprises an essential amino acid, a non-essential amino acid, fatty acid-free bovine serum albumin (BSA), insulin-transferrin-selenium (ITSG), β-mercaptoethanol, hypotaurine, 1-oleoyl-sn-glycero-3-phosphate (sodium salt) and salidroside.

The insulin-transferrin-selenium, β-mercaptoethanol, hypotaurine, 1-oleoyl-sn-glycero-3-phosphate (sodium salt) and salidroside are introduced to the culture system before the embryo development, so they can fully exert their positive effect as environmental factors to improve the production efficiency of bovine in vitro fertilized embryos and boost the development rate of bovine in vitro fertilized embryos, the number of blastocysts and the number of blastomeres, thereby significantly reducing the production cost of embryos.

In an embodiment, the culture medium comprises 0.1-2% by volume of the essential amino acid, 0.1-2% by volume of the non-essential amino acid, 1-10 mg/mL of the fatty acid-free BSA, 0.1-2% by volume of the insulin-transferrin-selenium, 10-100 μM of β-meraptoethanol, 1-10 mM of hypotaurine, 0.1-1 μg/mL of 1-oleoyl-sn-glycero-3-phosphate (sodium salt) and 0.01-0.2 mM of salidroside.

The supplement of essential amino acid, non-essential amino acid and fatty acid-free BSA can further improve the in vitro development quality of bovine embryos.

In an embodiment, the SOF basal medium comprises 261.68 mg/L of CaCl₂).2H₂O, 99.99 mg/L of C₆H₅Na₃O₇.2H₂O, 29.23 mg/L of glutamine, 372.18 mg/L of MgSO₄.7H₂O, 499.04 mg/L of inositol, 10.00 mg/L of phenolsulfonphthalein, 533.78 mg/L of KCl, 161.95 mg/L of KH₂PO₄, 44.02 mg/L of sodium pyruvate, 2.10025 g/L of NaHCO₃, 6.29399 g/L of NaCl and 0.757 mg/L of sodium lactate.

In an embodiment, the culture medium further comprises 12 μg/mL of gentamicin.

In a second aspect, this application provides a culture method for improving the development of bovine in vitro fertilized embryos and cloned embryos, comprising:

culturing the bovine in vitro fertilized embryos or the cloned embryos in the above-mentioned culture medium.

In an embodiment, the culture method comprises:

preheating the culture medium for at least 2 h; and

culturing the bovine in vitro fertilized embryos or the cloned embryos in the preheated culture medium in a CO₂ incubator for 7-8 days;

wherein the bovine in vitro fertilized embryos are obtained through 12-16 h of bovine sperm-egg binding; and the cloned embryos are somatic cell cloned embryos which are chemically activated with 6-DMAP in advance.

In an embodiment, the culture of the bovine in vitro fertilized embryos or the cloned embryos is performed at 38.5° C., 5% CO₂ and 7% O₂ under saturated humidity.

Compared to the prior art, the culture medium provided herein can effectively improve the development rate and quality of the bovine in vitro fertilized embryos and somatic cloned embryos.

### DETAILED DESCRIPTION OF EMBODIMENTS

The invention will be clearly and completely described below with reference to the embodiments. Obviously, described below are merely some embodiments of the invention, which are not intended to limit the invention. Other embodiments obtained by those skilled in the art based on the content disclosed herein without sparing any creative effort should fall within the scope of the invention.

Trypsin, gentamicin, cephalosporin, inorganic salt, paraffin oil (M8410), essentially fatty acid free (EFAF) BSA (Cat. No. A6003-5G), hypotaurine (Cat. No. H1384) and salidroside (Cat. No. SMB00072-1MG) are purchased from Sigma-Aldrich Inc.

Hepes-free M199 basal medium, essential amino acids (Cat. No. 1831512), non-essential amino acids (Cat. No. 1958909), insulin-transferrin-selenium (ITSG) (Cat. No. 1865342, insulin: 1 g/L; transferring: 0.55 g/L; selenium: 0.00067 g/L) and β-mercaptoethanol (Catalog number(Cat. No.): 1922445) are purchased from Thermo Fisher Scientific Inc.

Defined FBS is purchased from GIBCO Company.

1-oleoyl-sn-glycero-3-phosphate (sodium salt) (Cat. No. LP100-0025) is purchased from Enzo Life Sciences, Inc.

The SOF basal medium is self-prepared.

Unless otherwise specified, other reagents are all purchased from Sigma-Aldrich Inc.

Preparation of Ovum Collection Solution (PBS)

26.168 mg of CaCl₂.2H₂O, 37.218 mg of MgSO₄.7H₂O, 0.0203 g of KCl, 0.0200 g of KH₂PO₄, 0.0036 g of sodium pyruvate, 0.805 g of NaCl, 0.1153 g of Na₂HPO₄, 0.1 g of D-glucose, 6 mg of heparin sodium and 3 mL of FBS are mixed, diluted with deionized water to 100 mL and adjusted to pH 7.0-7.2 with 1 mM NaOH to prepare the ovum collection solution.

Preparation of Culture Medium for In Vitro Maturation of Oocytes

The 2-[4-(2-hydroxyethyl) piperazin-1-yl]ethanesulfonic acid (Hepes)-free M199 basal medium is added with 6 mg/mL of fatty acid-free BSA, 0.075 IU/mL of human menopausal gonadotrophin (HMG), 2 μg/mL of 17β-estradiol, 60 ng/mL of EGF, 40 ng/mL of bFGF, 50 μM of folic acid, 2 μg/mL of cholic acid and 50 ng/mL of CXCL12 to produce the culture medium for the in vitro maturation of oocytes.

Preparation of Synthetic Oviduct Fluid (SOF)

26.168 mg of CaCl₂.2H₂O, 9.999 mg of C₆H₅Na₃O₇.2H₂O, 2.923 mg of glutamine, 37.218 mg of MgSO₄.7H₂O, 49.904 mg of inositol, 1.000 mg of phenolsulfonphthalein, 53.378 mg of KCl, 16.195 mg of KH₂PO₄, 4.402 mg of sodium pyruvate, 210.025 mg of NaHCO₃, 629.399 mg of NaCl and 0.0757 mg of sodium lactate are mixed, dissolved and diluted to 100 mL with deionized water and adjusted to pH 7.2-7.4 with 1 mM NaOH to produce the SOF.

Preparation of SOFaa Solution

The SOF is used as basic medium and added with 1% by volume of essential amino acids, 1% by volume of non-essential amino acids, 6 mg/mL of fatty acid-free bovine serum albumin (BSA) and 12 μg/mL of gentamicin to prepare the SOFaa solution.

Preparation of mSOFaa Solution (the Culture Medium of the Invention for Improving the Development of Bovine In Vitro Fertilized Embryos and Cloned Embryos)

The SOFaa solution is used as basic medium and added with 1% by volume of ITS-G, 55 μM of β-mercaptoethanol, 2.5 mM of hypotaurine, 0.4 μg/mL of 1-oleoyl-sn-glycero-3-phosphate (sodium salt), 0.1 mM of salidroside, 10 ng/mL of progesterone, 100 μM of vitamin E, 1 mM of L-carnitine, 0.1 mg/mL of citrulline, 40 ng/mL of FGF, 10 ng/mL of corticosterone, 20 ng/mL of EGF, 25 ng/mL of insulin-like growth factor (IGF) and 20 ng/mL of LIF during the pre-heating to prepare the mSOFaa solution.

Preparation of Fertilization Medium

An inositol-free SOF is used as basic medium and added with 48 mg/mL of fatty acid-free BSA, 12 μg/mL of gentamicin, 63 μg/mL of L-arginine, 6 μg/mL of L-aspartic acid, 3 mM of L-carnitine, 2 mM of adrenaline, 20 mM of penicillamine, 10 mM of hypotaurine and 10 μg/mL of heparin to prepare the fertilization medium.

Preparation of Embryo Transfer Medium

The SOFaa solution is used as basic medium and added with 1% by volume of ITS-G, 100 μM of β-mercaptoethanol, 2.5 mM of hypotaurine, 0.4 μg/mL of 1-oleoyl-sn-glycero-3-phosphate (sodium salt), 0.1 mM of salidroside, 0.04 mg/mL of L-citrulline, 2.38 mg/mL of Hepes and 0.168 mg/mL of sodium bicarbonate during the preheating process to prepare the embryo transfer medium.

### Example 1 Preparation of Bovine In Vitro Fertilized Embryos

(1) In Vitro Maturation of Bovine Oocytes

Bovine ova were collected from a cow raised by Yangling Keyuan Clone Co., Ltd (collection time: January to December in 2019). Specifically, the ova in the follicles on the ovary of the cow injected with follicle-stimulating hormone (FSH) were collected by an ovum pick-up device, stored in a sterile collection solution at 38.5° C. and then transported to the laboratory within 0.5 h. The cumulus-oocyte complexes (COCs) were collected under a stereomicroscope and washed three times with the collection solution. Those COCs with normal oocyte morphology and complete granular cell were selected for the in vitro maturation (the number of the COCs for the in vitro maturation was defined as the total number of oocytes).

The selected COCs were washed twice with the culture medium for the in vitro maturation of oocytes and transferred to a 4-well plate containing 500 μL of the in vitro maturation medium, which was equilibrated at 38.5° C. in an incubator for 1 h in advance. Then the COCs were cultured at 38.5° C. and 5% CO₂ under saturated humidity for 20-22 h, and the mature COCs were blown repeatedly with a 1000 mL pipette to remove the diffused cumulus cells outside the oocytes. When there were only four to five layers of cumulus cells tightly coated outside the oocytes, the COCs were washed twice with the fertilization medium (BO medium) to remove the free cumulus cells, and the remaining COCs in which the oocytes were tightly coated with 4-5 layers of cumulus cells were transferred to a fertilization medium droplet which was equilibrated at 38.5° C. in an incubator for 2 h or more in advance.

(2) Thawing, Purification and Fertilization of Sperms

Cryopreserved sperms (collected from Xi'an Dairy Herb Breeding Center in October of 2013) were transferred from liquid nitrogen and thawed in a 37° C. water bath. A percoll gradient was prepared in a centrifugation tube with 2 mL of 90% Percoll at the bottom and 2 mL of 45% Percoll layered thereon. Then the thawed sperms were carefully placed on the 45% Percoll layer and centrifuged at 1,500 rpm for 20 min. About 200 μL of the semen at the bottom was carefully pipetted and added to the fertilization medium containing the oocytes. The fertilization system was cultured in an incubator for 20 h to perform the sperm-egg fusion.

(3) Culture of Fertilized Embryos

Experimental Group

500 μL of an mSOFaa solution (the culture medium of the invention) added with 2 mg/mL of hyaluronic acid was added to each well of a 4-well plate, covered with 500 μL of paraffin oil and pre-equilibrated at 38.5° C. and 5% CO₂ under saturated humidity for at least 2 h.

After the fertilization was completed, the remaining cumulus cells and sperms around the ova were removed with a mouth pipette or a pipette. The resulting presumptive bovine in vitro fertilized embryos were added to the mSOFaa solution in the 4-well plate (about 40 embryos per well) and cultured at 38.5° C., 7%02 and 5% CO₂ under saturated humidity for 72 h. The fertilized embryos without cleavage were picked out and removed, and the remaining embryos were continuously cultured in the incubator for 96 h (a total culture time of 168 h). The blastocysts with normal morphology were selected and transferred to an embryo transfer catheter containing the embryo transfer medium for embryo transfer. Whether the recipient bovine was pregnant or not was checked on the 33^{rd}-38^{th} day after the embryo transfer.

With regard to the control group, the fertilized embryos were cultured in the SOFaa medium, and the other operations were the same as the experimental group.

The development of embryos in the experimental and control groups was observed and recorded, and the results were shown in Table 1.

TABLE 1
Development of in vitro fertilized embryos

| | | Number of cleavage | Number of blastocysts |
|---|---|---|---|
| | Total | embryos (cleavage | on Day 7 |
| | number | rate %: number of | (rate %: number of |
| | of | cleavage embryos/total | blastocysts/number of |
| Group | oocytes | number of oocytes) | cleavage embryos) |
| Experimental | 110 | 62 (56.36%) | 21 (33.87%)^{b} |
| group | | | |
| Control | 114 | 59 (51.75%) | 12 (20.34%)^{a} |
| group | | | |

Notes:
different superscripts indicated significant difference; and the significance analysis was performed by chi-square test.

It can be seen from Table 1 that the culture medium of the invention can result in a higher proportion of blastocysts when used for the in vitro culture of embryos, which indicated that the culture medium provided herein can improve the in vitro embryo production efficiency.

The pregnancy rate was also calculated, and the results were shown in Table 2.

TABLE 2
Pregnancy rate of recipient bovines

| | Number of | Pregnancy rate on Day 40-45 |
|---|---|---|
| Group | recipient bovines | after embryo transfer |
| Experimental | 9 | 4 (44.44%)^{a} |
| group | | |
| Control | 19 | 6 (31.57%)^{b} |
| group | | |

Notes:
different superscripts indicated significant difference; and the significance analysis was performed by chi-square test.

It can be seen from Table 1 that the blastocysts obtained through the culture in the culture medium of the invention led to higher pregnancy rate after the transfer. In combination with Table 1, it was demonstrated that the culture medium of the invention can improve the in vitro embryo production efficiency.

### Example 2 Preparation of Bovine Somatic Cell Cloned Embryos

(1) Culture of Bovine Fetal Fibroblasts

A tube of bovine fetal fibroblasts (collected from a cattle farm owned by Yangling Keyuan Clone Co., Ltd) from 2^{nd} to 5^{th} generation of Holstein cows was transferred from liquid nitrogen, thawed at 39° C. and suspended with 0.8 mL of DMEM/F12 medium. The cell suspension was centrifuged, and the supernatant was discarded. Then the cells were resuspended with the cell culture medium, and 3 mL of the cell resuspension was inoculated to a culture dish with a diameter of 6 cm, which was subsequently cultured at 38.5° C. in a CO₂ incubator.

When the bovine fetal fibroblasts grew to a confluence of 80%, the culture medium was removed, and the cells were washed with a Ca²⁺ and Mg²⁺-free PBS, digested with a mixed solution of trypsin and EDTA and observed under an inverted microscope. When most of the cells contracted and rounded, and the intercellular space increased, the digestion was terminated with a DMEM/F12 medium containing 10% fetal bovine serum, and the cell suspension was blown with a pipette and centrifuged. The cells were suspended, inoculated into a 24-well plate at a ratio of 1:3 and cultured in the CO₂ incubator. The medium was replaced with a DMEM/F12 medium containing 0.5% fetal bovine serum two days before the preparation of somatic cell cloned embryos.

(2) Maturation of Oocytes

The selected COCs were washed twice with the culture medium for the in vitro maturation of oocytes and transferred to a 3 cm culture dish containing 3 mL of the in vitro maturation medium, which was equilibrated in an incubator for 1 h in advance. Then the COCs were cultured at 38.5° C. and 5% CO₂ under saturated humidity for 18-20 h, and the mature COCs were digested in normal saline with 0.1% hyaluronidase for 1-2 min and blown repeatedly with a 1000 mL pipette to remove the diffused cumulus cells outside the oocytes. After that, the remaining oocytes were washed three times with PBS and observed under the stereomicroscope, where those oocytes with a polar body were collected with a foreign body needle for use.

(3) Construction of Bovine Somatic Cell Cloned Embryos

Enucleation

Before the enucleation, the oocytes were incubated in a M199 medium containing 7.5 μg/mL of cytochalasin B, 4 mg/mL of fatty acid-free BSA and Hepes at 38.5° C. for 30 min. Under the micromanipulator, an enucleating tube with an inner diameter of 20 μm was employed to extract the first polar body and some of the surrounding protruding ooplasm.

Injection and Electrical Fusion

Bovine fetal fibroblasts with a size of 15-20 μm were selected and transferred into the zona pellucida of the enucleated oocytes. Then the reconstructed embryos were subjected to electrical fusion using a microelectrode. The reconstructed embryos were pre-equilibrated in the electrofusion solution for 3 min before the fusion, and the electrofusion was performed under a 150× micromanipulator. A top diameter of the two “Z”-shaped microelectrodes for fusion was 15 μm, and a rear end of each microelectrode was connected to the micromanipulator to render the membrane contact surface of the donor and acceptor of the reconstructed embryo perpendicular to the connecting line between the two electrodes. The fusion parameters were set as follows: voltage: 32V; pulse duration: 20 μs; pulse interval: 10 ms. The reconstructed embryos were observed under the microscope half an hour after the fusion, and the successfully-fused embryos were cultured in the culture medium for the in vitro maturation of oocytes for 3 h.

(4) Activation of Bovine Somatic Cell Cloned Embryos

3 h after the electrofusion, the bovine somatic cell cloned embryos were activated with ionomycin combined with 6-DMAP. Specifically, the cloned embryos were cultured in a SOFaa medium containing 2-5 μmol/L of ionomycin at room temperature for 4 min, washed 3-5 times with a SOFaa medium free of ionomycin and then cultured in a SOFaa medium containing 1-2 mmol/L of 6-DMAP at 38.5° C. and 5% CO₂ under saturated humidity for 4 h.

(5) In Vitro Culture of Bovine Somatic Cell Cloned Embryos

Experimental Group

500 μL of the mSOFaa solution (the culture medium of the invention) added with 2 mg/mL of hyaluromic acid was added to each well of a 4-well plate and then covered with 500 μL of paraffin oil. The 4-well plate was pre-equilibrated in a CO₂ incubator for at least 2 h. The activated bovine somatic cell cloned embryos were transferred to the above 4-well plate at 35-40 embryos per well, and cultured at 38.5° C., 5% CO₂ and 7% O₂ under saturated humidity.

The cloned embryos in the control group were cultured with a SOFaa medium, and the other operations were the same as those in the experimental group.

The development of cloned embryos was recorded and shown in Table 3.

TABLE 3
Development status of cloned embryos

| | | | | Number of blastocysts |
|---|---|---|---|---|
| | Total number | Number of cleavage | Number of blastocysts | of the grades A and B |
| | of oocytes | embryos (cleavage | on Day 7 | on Day 7 (rate %: |
| | for the | rate %: number of | (rate %: number of | number of blastocysts |
| | nuclear | cleavage embryos/total | blastocysts/number of | of the grades A and B/ |
| Group | transfer | number of oocytes) | cleavage embryos) | number of blastocysts) |
| Experimental | 133 | 97 (72.93%) | 30 (30.93%)^{b} | 17 (56.67%)^{b} |
| group | | | | |
| Control | 131 | 98 (74.80%) | 22 (22.44%)^{a} | 7 (31.81%)^{a} |
| group | | | | |

Notes:
different superscripts indicated significant difference; the significance analysis was performed by chi-square test; blastocysts of grades A and B were transplantable; blastocysts of grade C were not recommended for transplantation; the grading of blastocysts was determined according to the blastocyst quality scoring criteria published by International Embryo Transfer Association.

It can be seen from Table 3 that the culture medium of the invention can increase the ratio of cloned blastocysts when used for embryo culture, which indicated that the culture medium of the invention can improve the in vitro embryo production efficiency.

The description of the above embodiments is intended to enable those skilled in the art to implement and use the invention, and is not intended to limit the invention. Any modifications, changes and replacements made by those skilled in the art without departing from the spirit of the invention should fall within the scope of the invention.

What is claimed is:

1. A culture medium for improving development of bovine in vitro fertilized embryos and cloned embryos, comprising: a synthetic oviduct fluid (SOF) basal medium, citrulline, progesterone, vitamin E, L-carnitine, fibroblast growth factor (FGF), corticosterone, epidermal growth factor (EGF), insulin growth factor (IGF) and leukemia inhibitory factor (LIF).

2. The culture medium of claim 1, comprising: 1-100 ng/mL of progesterone, 10-1000 μM of vitamin E, 0.1-30 mM of L-carnitine, 1-100 ng/mL of FGF, 1-100 ng/mL of corticosterone, 0.01-1 mg/mL of citrulline, 1-100 ng/mL of EGF, 1-100 ng/mL of IGF and 1-100 ng/mL of LIF.

3. The culture medium of claim 1, further comprising: an essential amino acid, a non-essential amino acid, fatty acid-free bovine serum albumin (BSA), insulin-transferrin-selenium (ITSG), β-mercaptoethanol, hypotaurine, 1-oleoyl-sn-glycero-3-phosphate (sodium salt) and salidroside.

4. The culture medium of claim 3, comprising: 0.1-2% by volume of the essential amino acid, 0.1-2% by volume of the non-essential amino acid, 1-10 mg/mL of the fatty acid-free BSA, 0.1-2% by volume of the insulin-transferrin-selenium, 10-100 μM of β-mercaptoethanol, 1-10 mM of hypotaurine, 0.1-1 μg/mL of 1-oleoyl-sn-glycero-3-phosphate (sodium salt) and 0.01-0.2 mM of salidroside

5. The culture medium of claim 4, wherein the SOF basal medium comprises 261.68 mg/L of CaCl₂.2H₂O, 99.99 mg/L of C₆H₅Na₃O₇.2H₂O, 29.23 mg/L of glutamine, 372.18 mg/L of MgSO₄.7H₂O, 499.04 mg/L of inositol, 10.00 mg/L of phenolsulfonphthalein, 533.78 mg/L of KCl, 161.95 mg/L of KH₂PO₄, 44.02 mg/L of sodium pyruvate, 2.10025 g/L of NaHCO₃, 6.29399 g/L of NaCl and 0.757 mg/L of sodium lactate.

6. The culture medium of claim 5, further comprising: 12 μg/mL of gentamicin.

7. A culture method for improving the development of bovine in vitro fertilized embryos and cloned embryos, comprising:
culturing the bovine in vitro fertilized embryos or the cloned embryos in the culture medium of claim 1.

8. The culture method of claim 7, comprising:
preheating the culture medium for at least 2 h; and
culturing the bovine in vitro fertilized embryos or the cloned embryos in the preheated culture medium in a CO₂ incubator for 7-8 days;
wherein the bovine in vitro fertilized embryos are obtained through 12-16 h of bovine sperm-egg binding; and the cloned embryos are somatic cell cloned embryos which are chemically activated with 6-DMAP.

9. The culture method of claim 9, wherein the culture of the bovine in vitro fertilized embryos or the cloned embryos is performed at 38.5° C., 5% CO₂ and 7% O₂ under saturated humidity.
